(19) United States
(12) Patent Application Publication
(10) Pub. No.: US 2008/0234833 A1
(43) Pub. Date: Sep. 25, 2008

(54) Method of Designing and Manufacturing Artificial Joint Stem with Use of Composite Material

(75) Inventors: Shunichi Bandoh, Gifu (JP); Masaru Zako, Osaka (JP)

Correspondence Address: APEX JURIS, PLLC, 12360 LAKE CITY WAY NORTHEAST, SUITE 410, SEATTLE, WA, 98125, US

(73) Assignee: B.I. TEC LTD, Kakamigahara-shi, Gifu (JP)

(21) Appl. No.: 10/599,182

(22) PCT Filed: Mar. 23, 2004
(86) PCT No.: PCT/JP04/03977

§ 371 Date: Sep. 21, 2006

(51) Int. Cl. A61F 2/36; A61F 2/30

(52) U.S. Cl. 623/23.15; 623/18.11

(57) ABSTRACT

The method of designing and manufacturing the artificial joint stem, comprising steps of performing analysis of the internal stress of the artificial joint stem and bone and the adhesive stress of the artificial stem and bone, using the computer, based on the three dimension data indicating the structure of the bone formed by using plural tomographic images of the bone, the design condition involving the form and stiffness of the artificial joint stem configured by using at least one of the tomographic images and the three dimension data, wherein if the result of the analysis does not satisfy the design condition, the condition is changed to have the computer reanalyze and if the result of the analysis satisfies the design condition, the artificial joint stem is designed and manufactured with the stem data based on the result of analysis and the design condition.

### FIELD OF THE INVENTION

This invention relates to a method of designing and manufacturing an artificial joint stem as being implanted in a bone to form an artificial joint, particularly to the method of designing and manufacturing the artificial joint stem with the use of composite materials.

### BACKGROUND OF THE INVENTION

It has long been known that an artificial joint made to imitate a joint is implanted when a damaged joint is removed due to a broken bone. As one example of this artificial joint, FIG. 13 shows a structure of a conventional total hip prosthesis used for a hip prosthesis. This total hip prosthesis 100 is comprised of a socket 102 fixed to a pelvis 101, a spherical head 104 equivalent to a femoral head of a femur 103 and a stem 105 embedded in the femur 103.

As shown in the figure, the socket 102 and the head 104 make a pair and have a function of a spherical bearing. This socket 102 consists of synthetic resins such as high-density polyethylene, and the spherical head 104 comprises ceramics like zirconia or cobalt alloy. Such socket 102 and the head 104 have been improved in durability with many modifications in recent years so that they can maintain the functions longer than life expectancy of many patients who undergo total hip arthroplasty, and the focus has been shifted from the socket 102 and the head 104 to the stem 105 to prolong the life of the total hip prosthesis 100. The stem is often made of metal, and titanium alloy such as cobalt alloy and Ti6Al-4V is mainly used, considering the strength and effect on the human body.

As a method of fixing the stem to the femur, adhesive called cement-type has been used so far, and a cement-type total hip prosthesis stem using the method will be described below based on FIGS. 14-18.

FIG. 14 is a set of top views showing the examples of the conventional metal-made cement-type total hip prosthesis stem; FIG. 15A shows the condition before the cement-type total hip prosthesis stem is placed, and FIG. 15B is the section view, showing the condition in which the stem is placed in the femur. FIG. 16 is a cross section view of the internal structure of the epiphysis in the proximal side of the femur. FIG. 17 is an enlarged cross section view of the internal structure of bone. Also, FIG. 18A is a graph, showing the relationship between the modulus ratio of bone and the average porosity of bone, and FIG. 15B is a graph, showing the relationship between the thicknesswise compression ratio of bone and the average porosity of bone.

FIG. 14 shows various types of cement-type total hip prosthesis 105a-105d. These external forms are generally simple with straight lines, circles and circular arcs, and there are no problems although the external forms of the stems 105a-105d are simple because the adhesive is filled in the medullary canal constituting complex internal forms.

The method of fixing the cement-type total hip prosthesis stem to the femur 103 will be described below based on FIG. 15. First, spongy cancellous bone and bone marrow are removed from the medullary canal of the femur 103 with the use of a tool called broach, and an insertion hole 107 to insert the stem 105e is formed. Next, a bone plug 108 is embedded at the bottom of the insertion hole 107, and adhesive or cement 109 with two kinds of resin, base resin and hardener which are mixed at the predetermined ratio respectively is filled in the insertion hole 107 (see A). Then, the stem 105e is inserted in the insertion hole 107 and fixed to the femur 103 as the cement 109 hardens (see B).

In the epiphysis of the femur 103 where the stem is fixed, as shown in FIG. 16, the interior is fully filled with a spongy cancellous bone 110, and the cancellous bone 110 gradually decreases as approaching from the epiphysis 112 to the lower side of the diaphysis 113, and the interior of the diaphysis 113 is abbreviation cavities. Such bone structure is made by the force affecting as distributed loads on the spherical femoral head at the tip of the epiphysis 112 and is fairly rational in terms of dynamics.

Meanwhile, the interior of the compact bone 111 is the spongy cancellous bone 110 with more refined cavities as approaching toward the center of bone, and the cancellous bone 110 has a weaker structure than that of the compact bone 111.

Therefore, regarding the strength characteristic of bone, as shown in FIG. 18(A) and FIG. 18(B), as the average porosity of bone (cavity ratio per unit area) increases, its modulus of elasticity and compressive strength both decrease. For that reason, bone has a structure with decreasing modulus of elasticity and compressive strength as approaching toward the center away from the outer layer. As to the cement-type total hip prosthesis stem, the stem 105 is fixed to the femur 103 by impregnating the cement 109 within the refined cavities of the cancellous bone 110.

In this way, regarding the cement-type total hip prosthesis stem, the stem 105 is fixed to the femur 103 by hardening the cement 109, so the stem 105 can be fixed to the femur 103 for a fairly short time, which has an advantage in rehabilitating early for patients who undergo replacement operation with the total hip prosthesis 100. Therefore, it is particularly effective for elderly patients who are confined to bed for a long time and concerned with negative effects on other functions including motor function.

However, the cement-type uses two kinds of resin, base resin and hardener as the cement 109, and if they are not mixed enough, or the mixture ratio is inaccurate, unreacted monomer resin components which are not polymerized would remain and have harmful effects on the human body through the melt-out, and it is a source of causing various damages to the human body. Therefore, there is hesitation in using the cement-type to the youth with a long life expectancy.

Also, as to the cement-type, the stem 105 is fixed to the cancellous bone 110 of the femur 103 through the cement 109, and since the stiffness and strength of the cancellous bone 110 are not enough, the adhesive property to the stem 105 gets worse due to the weight of the stem 105, and the stem 105 gets loose or moves downward, called a sinking-down phenomenon. Especially when the sinking-down phenomenon occurs, the spherical stem 105 creates circumferential hoop stress like severing bone. Then, when the bone is cracked, patients suffer from the pain over a long period of time since there is no way to treat it so far.

As for the total hip prosthesis, the cement-type requires re-operation at a rate of five to twenty percent within ten years, but it is difficult to pull the stem 105 with the cement-type out of bone, and the re-operation itself is not easy.

Now, the cement-less type, fixing the stem 105 to the femur 103 without the use of cement 109, has been developed, and the following explains the conventional cement-less total hip prosthesis stem with the use of the cement-less type, based on FIG. 19-FIG. 21. FIG. 19 is top views showing the examples of the conventional cement-less type total hip prosthesis. FIG. 20(A) shows an enlarged view of the principal part of convex portion on the side of stem, and FIG.(B) is a fragmentary sectional view of the further enlarged sectional view. FIG. 21 is a sectional view of the conventional cement-less type total hip prosthesis stem fixed to the femur and cut in the axial direction, which is a different embodiment from that of FIG. 19.

As shown in FIG. 19, the conventional cement-less total hip prosthesis stem is made of metal such as titanium alloy which is the same as cement-type, and there are various forms in stems 105f-105j as shown in the figure, and as to the external forms of these stems 105f-105j, the part below neck 115 to fix the head 104 is somewhat bigger compared to the cement-type stems 105a-105e, but the forms as a whole are simple with the use of curves between straight lines. Compared to the cement type stems 105a-105e, the cement-less type stems 105f-105j have forms such that the gap between the external surface and internal surface of the insertion hole 107 of the stem 105 penetrated into the femur 103 narrows.

The cement-less type stem 105 is fixed to the femur 103, using growth of bone within the femur 103, and the gap between the internal surface of the insertion hole 107 and the external surface of the stem 105 narrows as the stem 105 is driven into the insertion hole 107 and bone grows from the internal surface of the insertion hole 107 toward the external surface of the stem 105, and thereby fixing the stem 105 to the femur 103.

As to this cement-less type stem 105, there is no adverse affect on the human body through the melt-out of the unreacted monomer in the cement 109 since the cement 109 is not used. Therefore, the cement-less type stem 105 can be also used to young patients. Moreover, in a re-operation because the stem 105 can be pulled out of bone with relative ease, it helps save trouble in re-operation.

However, the cement-less type fixes the stem 105 as bone grows, narrowing the gap between the bone and the stem 105, and it takes several months until the bone fills the gap, and the stem 105 is firmly fixed, and then patients need a rehabilitation period, which prolonged a period of patients' hospitalization, imposing a burden on patients. Moreover, due to a long period of hospitalization it was difficult to adopt the method to elderly people who were concerned with negative effects on other functions such as motor function.

Given this situation, in order for patients to rehabilitate, the convex portion 116 (concavity and convexity portion) is set up on the surface of the stem 105 so that the stem 105 can be fixed to the extent that patients do not have trouble living in the early stage of the postoperative period, and the stem 105 is mechanically connected to bone with the anchoring effect of the convex portion 116.

FIG. 20(A) and FIG. 17(B) are enlarged views of the convex portion 116 for the conventional cement-less type total hip prosthesis stem, and as shown in the figures, the stem 105 can be fixed to some extent in the early stage of patients' postoperative period as being mechanically connected to bone with concavity and convexity on the surface of the stem 105 and set-in structure of minute wedges or screws between the stem 105 and the bone. The size in the concavity and convexity of the convex portion 116 is very small, and various forms are suggested.

Moreover, in addition to the mechanical joint, a chemical joint method is also suggested as the convex portion 116, and for instance, crystal of hydroxyapatite, the main component of bone, is attached to the surface of the stem 105 with adhesive or the like, and the stem 105 is fixed to the femur 103 by chemically binding hydroxyapatite of the stem 105 and by growing bone. The one with either a mechanical joint or chemical joint, or both has been suggested.

In this way, by setting up the convex portion 116 on the cement-less stem 105, the initial fixation can be achieved to some extent in the early stage of postoperative period, which could relieve some of the burden from patients who were hospitalized for a long time.

However, in the case of the stem 105, it is hard to say the initial fixation was perfect, and in the case of these cement-less type stems 105f-105j, the joint between the stem 105 and bone is only partially connected to the compact bone 111 with high bone strength and mostly connected to the cancellous bone 110 with low bone strength, and thereby the joint strength between the stem 105 and bone being weak, and the stem 105 got loose by repetitive loads from the stem 105.

Also, the conventional stem 105 is made of metal such as cobalt alloy and titanium alloy, and because these alloys are difficult to cut, it is very hard to process the convex portion 116 with microscopic convexo-concave on the surface of the stem 105, which made the stem 105 very expensive.

Moreover, these alloys are excellent in corrosion resistance, and because it is difficult to apply adhesive surface treatment to the surface to form electrically neutral and stable oxide coating for adhesion of hydroxyapatite's crystal, the bonding strength of the hydroxyapatite is not stable and the hydroxyapatite exfoliates, which, as a result, creates a problem that the stem 105 gets loose.

Also, because the external form of the stem 105 is simple, it does not fit the internal form of the medullary canal, the load to the femur 103 is concentrated, and thereby becoming a source of pain and breakdown of bone through forcibly driving the stem 105 into the medullary canal. Regarding the elderly with weak bone strength and patients with osteoporosis, because they cannot bear such operation in which the stem 105 is driven into the femur 103 with a hammer, the cement-less stems 105f-105j could not be adopted.

In order to solve these drawbacks, a new cement-less type stem has been suggested. FIG. 21 shows the cement-less type stem, and the stem 105k is called custom made, and it is to provide the stem 105k having an external form which fits the internal form of the medullary canal 117 in the femur 103 of patients.

The custom-made stem 105k is taken pictures of each section in the two-dot chain line shown in FIG. 21 with a ultrasonic tomography photo device or the like, and numerical data is made, combining these images in three dimensions with three dimension CAD, and the external form of the stem 105k is processed based on the numerical data with a numerically-controlled processing machine (NC, CNC), and then the surface is finished by hand.

As shown in FIG. 21, because the external form of the stem 105k fits the internal form of bone, and the gap between the stem and bone is small, the stem 105k is fixed to bone in the early stage of the postoperative period, which can relieve patients' burden. Also, since the stem can be connected with the compact bone 111 with high bone strength, fixation of the stem 105 is strengthened, preventing the stem 105 from getting loose.

However, as to the custom-made stem 105k, as shown in the section perpendicular to the axis in FIG. 22, it proves that the part touching the internal surface of the medullary canal 117 is small in the circumferential direction. Especially the part of the epiphysis 112 of the proximal side of the femur 103 touching the internal surface of the medullary canal 117 is significantly small. On the other hand, the distal side, the contacting part is getting larger as approaching toward the diaphysis 113. Here, the proximal side of the femur 103 means the side of the hip joint, and the distal side means the side of the knee joint.

Although it tries to make the external form of the stem 105k fit the internal form of the medullary canal 117 as much as possible, the workability of the machine work for the external form of the stem 105k and the subsequent finish processing is required. To be more precise, generally when a three dimensional form is machined, the cutting tool for cutting the form uses a hemispheric-tipped ball-end mill, and with the ball-end mill, it cannot get a flat face only by the machine work, which leaves a trail like a furrow called sculpheight.

Therefore, it is necessary to smooth the surface by undercutting the sculpheight by hand after the machine work, but the stem 105 such as titanium alloy is difficult to cut, and the finishing requires very hard work. Therefore, the cement-less type stem made of titanium alloy became very expensive. Moreover, when convexo-concave is formed on the stem 105 to fit the internal form of the medullary canal 117, the finishing work would becomes difficult, it is too costly to adopt, and as the production time of stem 105 becomes longer the time a patient spends in the hospital becomes longer, which means the burden on patients cannot be relieved.

When designing the external form of the stem 105, one tries not to form convexo-concave on the surface, ensuring that the stem 105 does not get caught when inserting the stem in the medullary canal 117. Therefore, as shown in FIG. 22, because the internal form of the medullary canal 117 is complex in the proximal side of the femur 103, the external form of the stem 105k cannot correspond to the internal form, reducing the part that contacts with the stem 105k (see section Z1-section Z8-section in the figure). Meanwhile, because the internal form of the medullary canal 117 is simple in the distal side, it can easily correspond to the external form of the stem 105k, expanding the area that contacts with the stem 105k (see sections Z9-Z13 in the figure).

There is a term, Fit and Fill, to describe the relationship between the stem and the medullary canal. Fit means the contact ratio to the cortical bone, which is the ratio of the length of the cortical bone touching the stem to the entire circumference of the medullary canal in a section perpendicular to the axis of bone. Fill means the filling ratio in the medullary canal of the stem, which is the ratio of the section area of the stem to the area of the medullary canal in a section perpendicular to the axis of bone.

The higher Fit and Fill is, the better the accessibility of the stem and bone and the stronger force is transmitted from the stem to the bone. Therefore, as shown in FIG. 22, in the conventional stem 105k, Fit and Fill is low in the proximal side of the femur 103 and Fit and Fill is high in the distal side. The distal side where Fit and Fill is high receives more force coming from the stem 105k to the femur 103 and has a larger contacting area with bone, that is, the distal side where Fit and Fill is high is doing the supporting.

As shown in FIG. 16 and FIG. 17, the ossein that constitutes the compact bone 111 and the cancellous bone 110, that is the trabecular bone, is formed to continuously extend to a particular direction, its strength increased in this particular direction and thus in the structure of orthotropic anisotropy. This structure is similar to that of bamboo and wooden board of straight grain. This trabecular bone extends out from bone's external form to the internal side in the epiphysis part 112, but in the diaphysis 113 the trabecular bone is formed along with the external form. Here, in contrast to the superior ability to transmit perpendicular and torsional loads in the bone surface of relatively weak compact bone 111, it is difficult to transmit a load from the stem in interior cancellous bone 110 of other bones.

Therefore, it is desirable to stabilize stem in the epiphysis (proximal side) of compact bone 111. That is, the best relationship between the stem and the medullary canal is expected in such ways that the fit and fill is high in the epiphysis section (proximal side). Hereafter, the fixing in the proximal side and the fixing in the distal side are called the proximal fixing and the distal fixing respectively.

However, as shown in FIG. 22, the fit and fill is low in the proximal side and the contacting area is small, and thus there are areas where force from the stem 105k is applied to bone and other areas where the force is not applied, which results in stress shielding. This stress shielding, deriving from bone's physiological behavior, is a phenomenon in which bone thickens in sections where force is applied and, conversely, bone becomes thin in sections where force is not applied. In this way, bone becomes thin in the sections where force from the stem 105k is not applied, reducing the conjugation with the stem 105k and causing the stem 105k to become loose.

Also, as shown in FIG. 22, the stem 105k turns easily in the stem 105k because the contacting area between bone and the non-circular cross section in the proximate side—that is, the section matching the internal form of the medullary canal 117—is minimal, and because the cross section is a near circular form in the distal side. As a result, rotation and fixation of the stem 105k was not satisfactory.

Moreover, stainless alloy such as high-corrosive-resistant cobalt alloy and titanium alloy are used in the above-mentioned stem 105. If the high-corrosive-resistant oxide film is removed through abrasion of the surface of the stem 105 by micro motion in the contacting area with bone resulting from the stainless alloy being embedded in the body for a long period of time, a micro opening called a corrosion pit is generated from the body fluid because the salinity in the body is the same as that of seawater. There has been a case reported in which a corrosion pit caused metal fatigue and fractured the stem.

As such, various materials are suggested as the stem's raw material to replace metals. Some composite materials are among the suggestions. FIG. 23 indicates the nature of the strength (fatigue strength) of the composite materials. First, while the fatigue strength of the titanium alloy 118a decreases gradually as a load is repeatedly applied, the composite material 119, especially in the case of the carbon fiber reinforced plastic (CFRP), has excellent durability, in which its fatigue strength rarely decreases even if a load is repeatedly applied. The symbol 118b, shown by the dotted line in the figure, indicates titanium alloy when it is macerated in seawater.

For example, it has been suggested to make the center of the stem metallic and wrap its outer side with composite materials such as FRP (fiber reinforced plastic). In U.S. Pat. No. 4,892,552, Japanese Unexamined Patent Publication No. 5-92019, and Published Japanese Translations of PCT International Publication No. 7-501475, it is suggested to manufacture the stem using the carbon fiber reinforced plastic. The stems in these proposals attain the same stiffness as metal by using carbon fiber reinforced plastic. By embedding fibers in resin harmless to human body there is no melt out of harmful substances into the body as with metal.

However, none of the above inventions have been in practical use in the current status. That is to say, the above inventions to make the center of the stem metallic and its external side wrapped around with FRP have ended in failure since the stem becomes loose in the early postoperative period, resulting from micro motion between the FRP and bone or between the FRP and the center of the metallic section. The cause of this failure is thought to be the fact that the stem's bending stiffness only applies to the center of the metallic section, making the overall bending stiffness low, and the distribution of stress in the area contacting bone is concentrated at both ends, leading to the occurrence of micro motion since the stem cannot resist the stress.

Also, U.S. Pat. No. 4,892,552 claims a sheet-shaped laminate made from carbon fiber impregnated with resin that has coupons cut out in a way such that the carbon fiber's direction is parallel to the external form and other coupons cut out in such a way that the carbon fiber's direction is 45°, these two types of coupons are piled up alternately, heat and pressure are applied to form a bloc, and the stem is manufactured by machining in which the bloc is scraped off. This merely substitutes metal with the composite material. While avoiding harmful substance melt out, it does not solve any other problems.

Furthermore, the Unexamined Patent Publication No. 5-92019 claims the stem having the first-direction strength support with reinforcing fiber in the longitudinal direction of the stem outside of the intermediate part that is hollow and the second-direction strength support with reinforcing fiber in the 45° direction from the longitudinal direction of the stem further outside. In this stem, the first-direction strength support deals with bending stiffness and the second-direction strength support deals with torsional stiffness with a structure utilizing the characteristics of a composite material. However, the second-direction strength support located outside the stem is manufactured by wrapping the strip-shaped reinforcing fiber. With this method it is difficult to attain the external form that fits the internal form of the medullary canal, necessitating the coating layer further outside of the second-direction strength support, and the stem may get loose since the stress is concentrated in both ends of the coating layer.

Moreover, in Published Japanese Translations of PCT International Publication No. 7-501475, carbon fiber reinforced plastic having carbon fibers embedded in the thermoplastic polymer is used as a stem, and stiffness of the stem is changed as varying the wrap angle of that carbon fiber from area-to-area of the stem; however, also this stem, because the external form is formed by wrapping carbon fiber, a concave form cannot be formed in the circumferential direction (fiber direction of the carbon fiber) of the stem, and it is difficult to attain the external form that fits the internal form of the medullary canal and initial fixation of a stem that raises the fit and fill cannot be achieved.

The problem was concentration of stress caused by connecting the stem and bone. FIG. 24 explains the concentration of stress in a patterned form. FIG. 24(A) indicates the condition of stress on the adhesive joint when members of the same stiffness are glued together. In this situation, the average stress applied on the adhesive joint between the member 120 and the member 121 is smaller than the simplified average stress calculated by simply dividing the compressive loading by the adhesive area, and the stress is applied mainly on both ends of the adhesive joints (indicated with a dashed line in the figure). On the other hand, the compressive stress of the member 120 and the member 121 gradually decreases by shear stress applied to the adhesive joint as getting toward the left in the figure and becomes zero at the left-end section (indicated with dashed lines in the figure).

Also FIG. 24(B) indicates the condition of stress on the adhesive joint when members of different stiffness are adhered. In this example, the member 121 of (A) is replaced by the member 122 with high stiffness. The stress is particularly concentrated at the right-end section of the adhesive joint, and the degree of stress is greater than that of (A) (indicated with dashed lines in the figure). Also, compressive stress is drastically reduced from the right-end section of the adhesive joint (indicated with dashed lines in the figure). We know from the above that the loading is transferred intensively at the one end of the adhesive joint when one member's stiffness is high.

Furthermore, FIG. 24(C) indicates the condition of stress on the adhesive joint when the length of adhesive joint in the example FIG. 24(B) is shortened. In this case, the average stress applied to the adhesive joint increases to the extent the adhesive area becomes smaller, yet the amount of stress concentration decreases and the total stress concentration does not change (indicated with dashed lines in the figure). Also, while compressive stress drastically decreases from the right-end section of the adhesive joint, high stress is maintained through the left-end section to the extent the adhesive section shortens (indicated with a dashed lines in the figure).

As shown in FIG. 24(A) and FIG. 24(B), we know hat the stress is concentrated at the end points of the adhesive section. That is, the stress concentration occurs at the both ends of connecting the point between the stem and bone. In particular, when comparing the stiffness of the stem and bone, the metallic stem made from titanium alloy is equivalent to the example in FIGS. 24(B) and (C) since its stiffness is greater than that of bone, and a high loading concentration applies at the ends of the connecting section, starting the separation of the stem and bone from this section which leads to the stem to become loose.

Given the above factors, the method in FIG. 24(D) can be considered as a method to alleviate the occurrence of stress concentration at the ends of the adhesive joint. For the member 123, the taper section 124 is provided on the side opposite of the adhesive joint of the member 123, varying the thickness in the middle of the connecting section. As such, the stiffness of the member 123 decreases on the way to the right-end section, and extended to the right-end section while keeping the stiffness low. In this case, stress concentration drops drastically, becoming close to the average stress of the adhesive joint (indicated with dashed lines in the figure). Also, the distribution of compressive stress is similar to FIG. 24(C) (indicated with dashed lines in the figure). Making the member 123 in such a form may reduce overall adhesive stress while keeping the member's overall compressive stress.

As a result, in the example of FIG. 24(D), the stress concentration is reduced while concentrating the stress at the adhesive section other than the ending points, and thus the separation of the adhesive section can be controlled even if the stress is concentrated.

That is, making the relationship between the stem and bone like FIG. 24(D) enables the stress concentration at the diaphysis to be transferred to epiphysis, and to control the occurrence of stress shielding since a high compressive stress is maintained at the adhesive section in its entirety. Also, the adhesive section is equivalent to the cancellous bone, and the separation of the cancellous bone from the stress concentration can be controlled at the end points of the connecting section with the stem.

As such, it is known in the traditional stem 105 that a porous coating of titanium alloy is applied on the proximal side surface of the stem 105 in order to increase the conjugation of bone in the proximal side, and that fixing is not to be done on the distal side by reducing the conjugation with bone through mirror finishing the tip part of stem 105 located on the distal side.

However, the conventional system is manufactured from materials that are difficult to cut such as titanium alloy, and it was impossible to process in the hollow section, and thus the method in FIG. 24(D) cannot be applied to the conventional metallic stem.

In the example in FIG. 24D, the member's thickness is varied as a means to change the stiffness. But for the composite material, the stiffness can also be changed by changing the direction of the reinforced fiber, in addition to the thickness of the member, thereby allowing the changes in both thickness and direction of the reinforced fiber.

As mentioned above, according to the invention, one can provide the method of designing and manufacturing the artificial joint stem with the use of composite material that may be made in a short period of time with a lower cost, which connects bones without using cement, not getting loose for a long period of time, excellent in the durability, and is provided with the stiffness and the external shape appropriate for each patient.

### SUMMARY OF THE INVENTION

In order to resolve the problems raised above, the method of designing and manufacturing the artificial stem with the use of the composite materials relating to this invention is configured to provide the structure of the method comprising steps of performing, as using a computer, an analysis involving an internal stress of the artificial joint stem and an adhesive stress of the artificial joint stem and a bone based on three dimension data indicating a structure of the bone made by using plural bone tomographic images and a design condition involving a form and stiffness of the artificial joint stem configured at least by one of the tomographic images and the three dimension image; having the computer to reanalyze as changing the design condition if a result of the analysis fails to satisfy the design condition; designing and manufacturing the artificial joint stem using stem data based on the result of the analysis and the design condition if the result of the analysis satisfies the design condition.

Here, for example the fiber reinforced resin can be used as the composite material. Carbon fiber, ceramic fiber, glass fiber, and aramid fiber can be examples of that reinforcing fiber. The ceramic fiber having a titanium component with silicon carbide as a main part, such as the product named “tirano fiber” is an example of a ceramic fiber. For turning those fibers into a continuous fiber, one can use filaments, blind form, woven fabrics, and non-woven fabrics, or for short fibers, one can use a chop shape. Carbon fibers are preferable and high modulus carbon fiber is the most preferable among them. As for the resin, examples include polyether ether ketone, polyetherimide, polyether ketone, polyacryl ether ketone, polyphenylene sulfide, polysulfone. The most preferable is a thermoplastic resin that is harmless to the human body and does not melt out. In order to increase flexibility at the time of laminating, it is also acceptable to use it in a fiber shape or sheet shape Furthermore, it is also acceptable to use woven fabrics formed by the above-mentioned reinforcing fiber and the above-mentioned resin when molding an artificial joint stem.

Also, as for the device for obtaining cross sectional images, the device can be but is not limited to any commonly known cross section imaging device; for example, a nondestructive tomography scanner such as CT or MRI. Furthermore, use of a device for obtaining cross-sectional images by measuring the difference in transmission speed in the dislocated part is desirable. If this type of device is used, that transmission speed can be used as data and based on that transmission speed the stiffness of the bone (Young's modulus) can be derived. For example, Young's modulus of bone as illustrated in FIG. 18(A) to FIG. 8(B) and density and derivation of that relationship, by combining the transmission velocity obtained from that relationship in cross sectional images, Young's modulus can be obtained in all areas of the bone, thus it becomes possible to analyze rigidity of the entire bone based on the obtained Young's modulus.

Furthermore, examples of the design condition may be the external form of the artificial stem (hereinafter sometimes simply called stem) based on the patient's tomographic image and the three dimension image created by the three dimension data based on the image and stiffness and strength determined at the respective portion/area of the stem, wherein the design condition is configured as including the doctor's treatment plan for the patent.

According to this invention, this invention makes it possible to create the three dimension data including the bone structure form plural tomographic image, to analyze various stress using the computer based on the design condition of the three dimension data and stem, repeat the changes and analysis of the design condition until the result of the analysis satisfies the design condition, create the stem data of the stem with appropriate form and stiffness, and design and manufacture the stem based on the stem data, thereby enabling to design and manufacture the stem with the form and stiffness in correspondence to the patient's bone shape and structure. As a result of the fit and fill of the stem and bone is increased to make the initial fixation possible and to raise the rotational fixation, an early discharge from the hospital is possible through shortening the hospitalization period, and an early social rehabilitation is possible and thus relieving the burden on the patient. Also, this method can be utilized for senior people, who have concerns about adverse effect of motor functions and other functions resulting from a long-term hospitalization.

Also, because fit and fill can be increased, the stem can be well connected to bone without the use of cement, and there is no adverse affect on the human body through the melt-out of the unreacted monomer from not being mixed enough or an inaccurate mixture ratio of cement.

Furthermore, because the stem can have the stiffness distribution corresponding to the patient's bone stiffness distribution, the load from the stem to the bone can be transmitted without deviation, and generation of stress sealing is controlled, thereby preventing form weakening the connection between the bone and stem and loosening the stem and improving the durability of the stem.

Also, the computer is used to perform the complicated three dimension stress analysis using the finite element method, which enables to shorten the time necessary for this analysis dramatically, shorten the time necessary for the manufacturing process dramatically, and also reduces the burden on the patient with respect to the hospitalization. The time can be further reduced by using digital data images in the cross-sectional images.

Also, the composite material used in the stem, in particular, by using composite material that is harmless to the human body, there is no adverse affect to the human body unlike the conventional metallic stem in which substances harmful to the human body melt-out from the stem to the inside of human body. Also, the composite material is excellent in formability and workability compared to titanium alloy, and the desired form can easily be attained. Along with the cost of that being low, it also becomes possible to manufacture a stem in a short time.

The method of designing and manufacturing the artificial joint stem with uses composite materials regarding this invention may be structured such that an external form of an epiphysis approximately fitting an internal form of an insertion hole formed in said bone, said artificial joint stem has a main part with stiffness around a boundary between epiphysis and diaphysis varies so as to lower the stiffness as approaching the diaphysis and a neck to place a spherical head in the artificial joint thereon.

Here, as the insertion hole formed in the bone, for example, although the prescribed internal form into the patent's bone by the computer controlled surgical robot using the above stem data in this example, the insertion hole may be formed by the broach cutter in another example.

Furthermore, as for the method of changing the stiffness of the stem's main part, the stiffness can be changed, for example, by formulating the stem with the composite material with the prescribed thickness and making the thickness thinner as approaching from the epiphysis area to the diaphysis area, or the stiffness can be changed by changing the fibrous direction of the reinforced fiber included in the composite material. These methods can be performed independently or in any combination thereof, and the combination is not limited as long as possible to change the stiffness.

According to the present invention, in addition to the above-effects, the stem's external form fits the internal form of the insertion hole that is penetrated into bone, and the stem can be fixed without slamming the stem into the insertion hole with a hammer, and the stem can be utilized for osteoporosis patients and elderly people whose bone's strength is weak.

Also, because the stem's external form in the epiphysis area fits the internal form of the insertion hole that is penetrated into bone, fit and fill can be high, and the stem can be fixed in the epiphysis area. That is, using an example of the femur, as the epiphysis area, the stem can be fixed near the femur, which means the proximal fixing is possible, transferring the loading well from the stem to bone.

Also, in the proximity of the boundary between the epiphysis area and the diaphysis area, the stiffness of the stem's main part varies in such a way that the stiffness becomes low as approaching toward the diaphysis. As a result, the stress concentration at the ends of the connecting section between the stem's main part and bone can be controlled, and the stem getting loose because of the stress concentration that breaks away the connecting section can be prevented. Also, since the stiffness in the diaphysis area is made low, the stem's loading is mainly transferred to the epiphysis area. If applied to the femur, for example, the proximal fixing, in which the force is transferred in the epiphysis area that is the proximal side, can be done.

The method of designing and manufacturing artificial joint stem with the use of composite material further comprising “a guide section, provided at the tip of the main part and placed at the disphysis, the guide section has a lower bending and stretching/tensile stiffness than the main part.”

According to the invention, the guide section is provided in the forefront of the stem, and as a result, the stem can be easily inserted in the insertion hole during the operation when inserting the stem into the insertion hole penetrated into bone because the stem's insertion is guided by the guide section.

Also, since the bending and tensile stiffness of the guide section is made lower than the main part, the stress applied to the connecting section between the guide section and bone can be less than the main part. To explain in detail, this invention, as having the same structure of the example in FIG. 24D, the stress concentration at the ends of the connecting section between the stem's main part and bone can be controlled, and may prevent the stem from getting loose due to the stem's separation from bone. Also, the stem's loading is transferred from the guide section to bone via the main part, thus for the femur, for example, it is the proximal fixing and the stem's loading can be well transferred to bone. Furthermore, also at the guide section, the stress shielding can be controlled for bone contacting the guide section, since the compression stress is equally applied.

The method of designing and manufacturing artificial joint stem with the use of composite material in the invention can also have a composition that “the computer performs analysis including the internal stress of the bone by using a finite element method.” Here, the finite element method is a known structure analysis method wherein the subject for analysis is broken down into simple shape-elements such as triangle and rectangular and the respective element is calculated to perform analysis. Furthermore, as shown in FIG. 16, because the internal bone system is not uniform, for example, the analysis may be performed as allocating the predetermined number per element according to the density, and the respective value can be automatically allocated by the predetermined method.

According to this invention, the stress analysis is performed by using the finite element, and therefore, time necessary for analysis can significantly be shortened and the result of the analysis can become closest possible to the characteristics of the actual bone, thereby increasing the reliability of the analysis result.

The method of designing and manufacturing artificial joint stem with the use of composite material in the invention can also have a composition that “the numerical control forming device or the processor is controlled based on the stem data to form the model of the artificial joint stem or the forming die.”

Here, for example, the numerical control forming device may be a light forming device or a laser forming device which hardens such as light hardening resin using visible laser beam and infrared rays and dissolves the work piece, and for example numerical control (NC) device, computer numerical control (CNC) processor, or machining sensor processor can be examples of the numerical control processor.

According to this invention, because the model of the artificial joint stem or a forming die is made by using the numerically controlled forming device or processor, based on the stem data, devices such as the forming device can easily be controlled, thereby reducing the number of manufacturing steps for the model or forming die and increasing the dimensional accuracy.

Also, the forming die of the stem is sufficient if it lasts one time, a material of which preferably gives high heat resistance for forming composite materials as well as excellent release characteristics and economical efficiency, and for example the material may be selected from such as gypsum, resin, fused salt, aluminum alloy, and low melting point alloy as necessary. Furthermore, when the stem model is formed, the forming die is made by reverse moulage from the model, and the moulage material can be selected from the above identified group as necessary.

The method of designing and manufacturing artificial joint stem with the use of composite material in the invention can also have a composition that “the automatic cutter is controlled based on the stem data to obtain the material for the composite material when forming the above described artificial joint stem.”

This invention allows to obtain the material for the composite material by the automatic cutter using the stem data, thereby preventing from causing mistakes in sizing as obtaining the material and reducing the time of obtaining the material.”

The method of designing and manufacturing artificial joint stem with the use of composite material in the invention can also have a composition that “a laminating position of the composite material used when forming the artificial joint stem is displayed on the forming die of the artificial joint stem based on the stem data.”

According to this invention, the laminating position is displayed on the forming die of the stem as irradiating the laser beam thereon, which enables to prevent from causing mistakes in the determination of the laminating position and laminating order and to manufacture the stem as meeting the design conditions such as the desirable stiffness.

As mentioned above, according to the invention, one can provide the method of designing and manufacturing the artificial joint stem with the use of composite material that may be made in a short period of time with a lower cost, which connects bones without using cement, not getting loose for a long period of time, excellent in the durability, and is provided with the stiffness and the external form appropriate for each patient.

### BRIEF DESCRIPTION OF THE DRAWINGS

The above and other objects, features, advantages and technical and industrial significance of this invention will be better understood by the following detailed description of the preferred embodiments, when considered in connection with the accompanying drawings, in which:

FIG. 1A is a front view of the artificial joint stem manufactured with the use of the method of designing and manufacturing the artificial joint stem using the composite material in the invention, and FIG. 1B is its side view thereof;

FIG. 2A is a cross section view taken along the line A1-A1 of FIG. 1, and FIG. 2B is a cross section view taken along the line A2-A2 of FIG. 1;

FIG. 3 is a cross section view of each B1-B6 in FIG. 1 at the respective height that are cut in each level perpendicular to the axes;

FIG. 4A is a cross section view showing the enlarged structure of the surface treatment section, and FIG. 4B is a cross section view of the further enlarged B part shown with an arrow in FIG. 4A;

FIG. 5 is a block view of the functional structure of the computer in the method of designing and manufacturing the artificial joint stem using the composite material of this invention;

FIG. 6 is a flow chart of the process summary of the method of designing and manufacturing the artificial joint stem using the composite material of this invention;

FIG. 7A shows multiple tomograms, where FIG. 7B is a view showing the condition of reading the form as the two dimensional data and FIG. 7C is a view showing the condition of the element breakdown after making three dimensions;

FIG. 8A is a view showing the rough element breakdown of the bone, FIG. 8B is an explanatory view illustrating the method for obtaining the stiffness of the bone, and FIG. 8C is a view showing the detail element breakdown of the internal portion of the bone;

FIG. 9A is a graph of the contact ratio to the cortical bone and the filling ratio in the medullary canal of the stem in FIG. 1, FIG. 9B is a graph of bending and tensile stiffness, and FIG. 9C is a graph of torsional stiffness;

FIG. 10A is a front view of the artificial joint stem different from the one in FIG. 1 manufactured with the use of the method of designing and manufacturing the artificial joint stem using the composite material in the invention, and FIG. 10B is its side view. thereof:

FIG. 11 is a set of cross section views of C1-C6 in FIG. 10 that are cut in each level perpendicular to the axes;

FIG. 12A is a graph of the filling ratio in the medullary canal of the stem in FIG. 10.

FIG. 12B is a graph of bending and tensile stiffness, and FIG. 12C is a graph of torsional stiffness;

FIG. 13 is a view showing the structure of the conventional total hip prosthesis;

FIG. 14 is a set of top views showing the examples of the conventional metal-made cement-type total hip prosthesis stem;

FIG. 15A is a view showing the condition before the cement-type total hip prosthesis stem is placed, and FIG. 15B is a cross section view showing the condition in which the stem is placed in the femur;

FIG. 16 is a cross section view of the internal structure of the epiphysis in the proximal side of the femur;

FIG. 17 is an enlarged cross section view of the internal structure of bone;

FIG. 18A is a graph showing the relations between the bone's modulus ratio and the average porosity of bone, and FIG. 18B is a graph showing the relations between the thicknesswise compression ratio of bone and the average porosity of bone;

FIG. 19 is a set of top views showing the examples of the conventional cement-less type total hip prosthesis;

FIG. 20A is an enlarged view showing the principal part of convex portion on the side of stem, and FIG. 20B is a fragmentary sectional view of the further enlarged sectional view;

FIG. 21 is a set of cross section views of the conventional cement-less type total hip prosthesis stem fixed to the femur and cut in the axial direction, which is a different embodiment from that of FIG. 19;

FIG. 22 is a set of cross section views of Z1-Z13 in FIG. 21 that are cut in each level perpendicular to the axes;

FIG. 23 is a graph showing the change of fatigue strength by cyclic loading of composite material and titanium alloy;

FIG. 24A is a view showing the condition of stress on the adhesive joint when members of the same stiffness are glued together, FIG. 24B is a view showing the condition of stress on the adhesive joint when members of different stiffness are glued together, FIG. 24C is a view showing the condition of stress on the adhesive joint when the length of the adhesive joint of the example in FIG. 24B is shortened, and FIG. 24D shows the condition of stress when the stiffness of either member is changed on the way;

### DESCRIPTION OF THE PREFERRED EMBODIMENTS

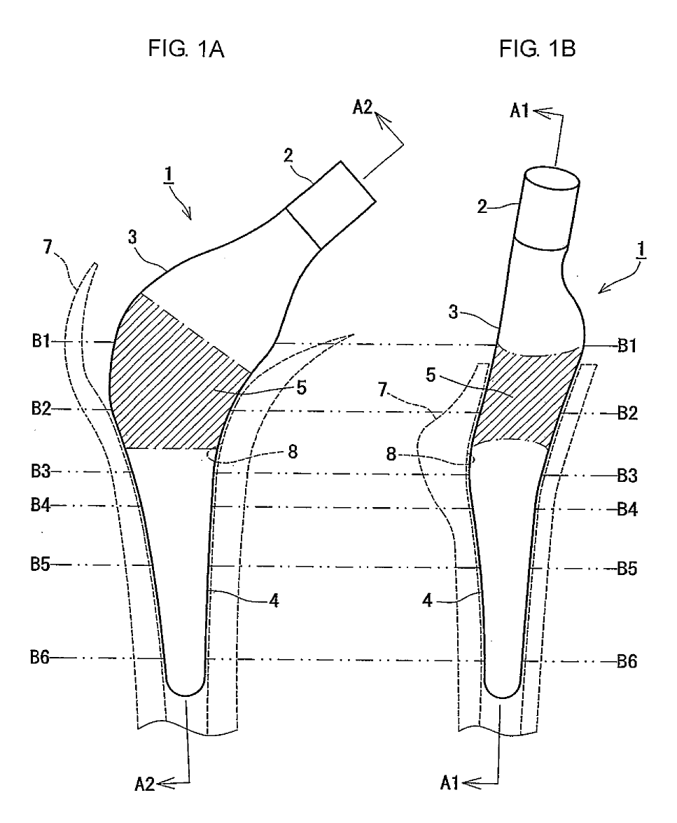

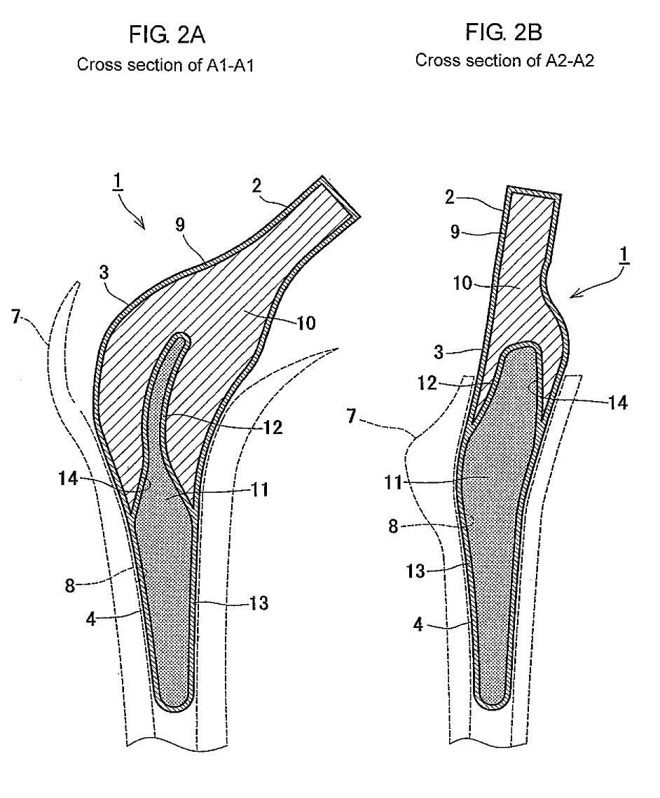

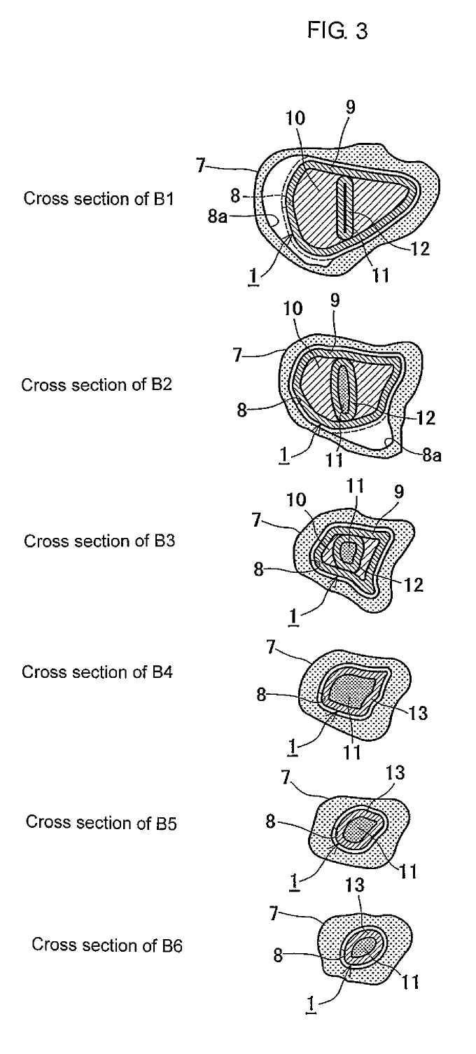

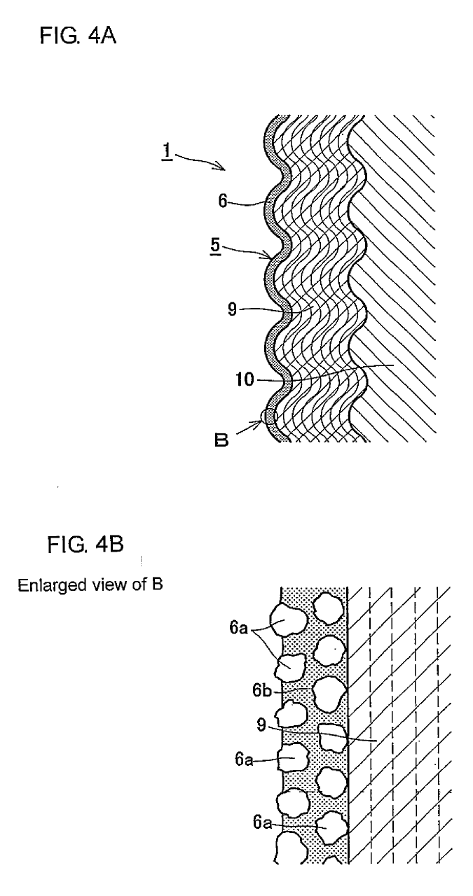

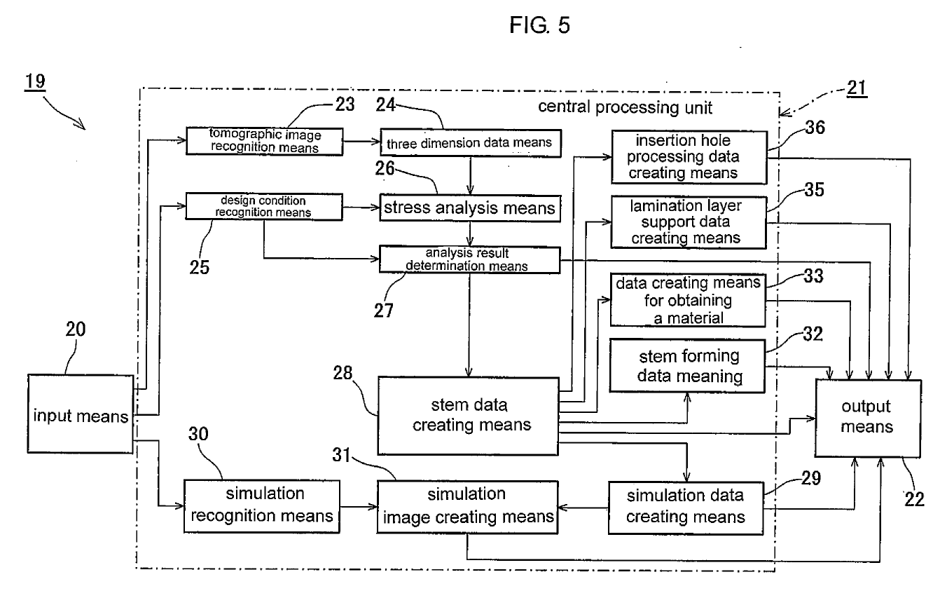

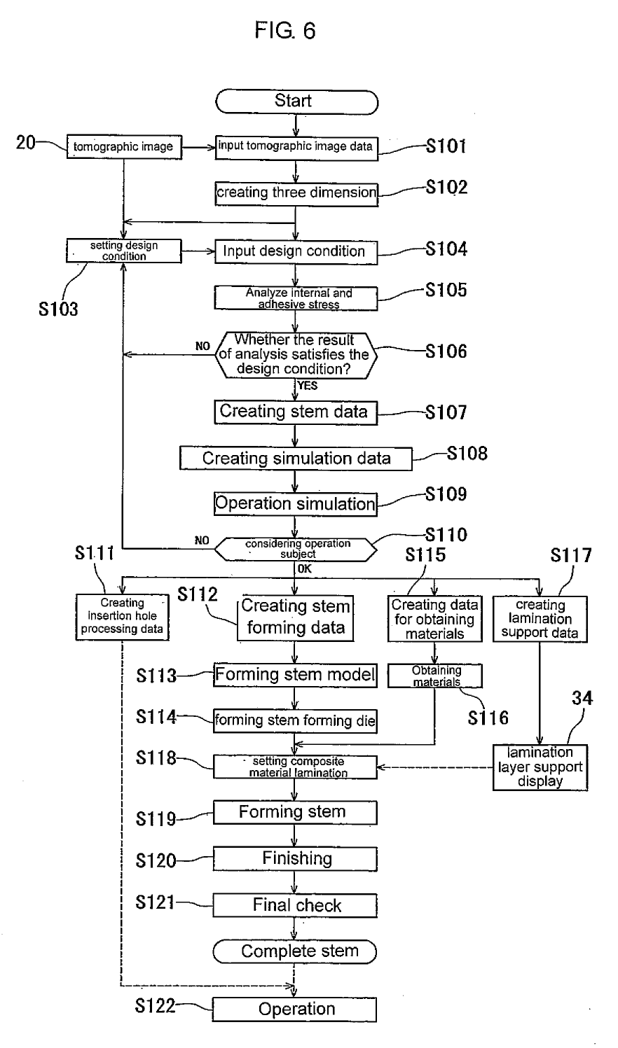

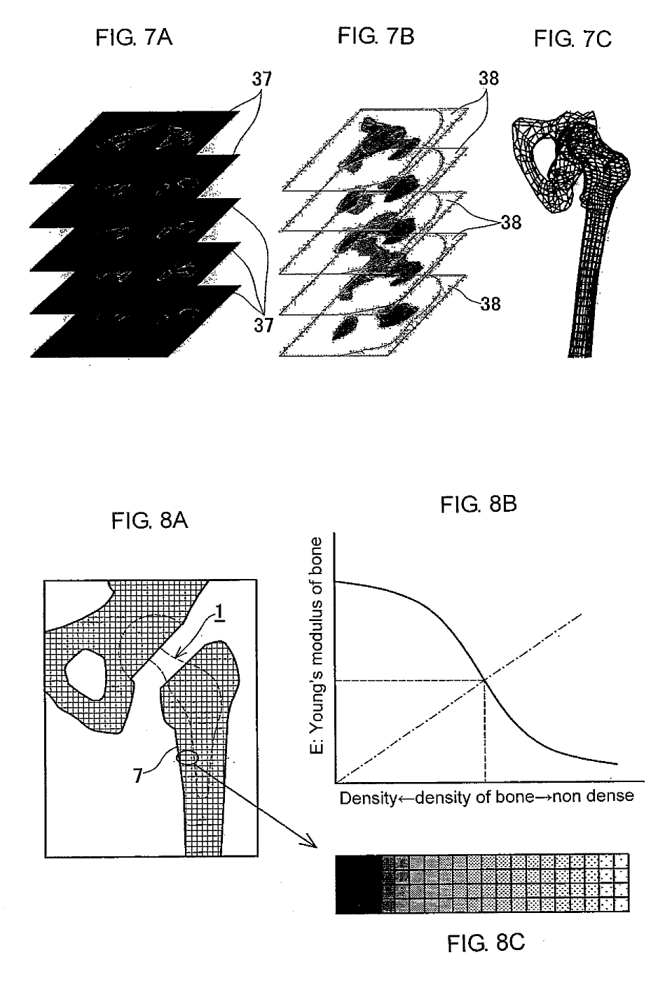

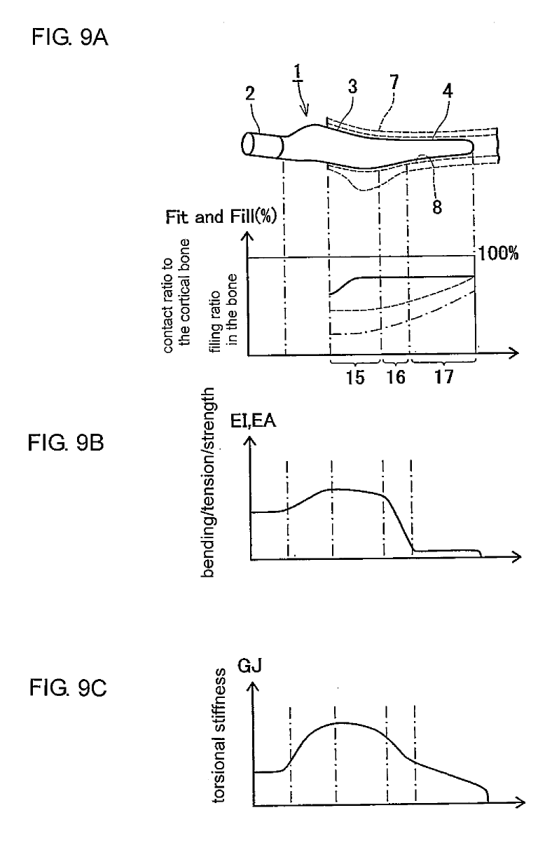

Below, the preferred embodiments are illustrated in details based on the FIGS. 1-9. FIG. 1A is a front view of the artificial joint stem manufactured with the use of the method of designing and manufacturing the artificial joint stem using the composite material in the invention, and FIG. 1B is its side view thereof. FIG. 2A is the section view taken along the line A1-A1 of FIG. 1, and FIG. 2B is the section view taken along the line A2-A2 of FIG. 1. FIG. 3 is a set of cross section views of B1-B6 in FIG. 1 at the respective height that is cut in each level perpendicular to the axes. FIG. 4A is a cross section view showing the enlarged structure of the surface treatment section, and FIG. 4B is a cross section view of the further enlarged B part shown with an arrow in FIG. 4A. FIG. 5 is a block view of the functional structure of the computer in the method of designing and manufacturing the artificial joint stem using the composite material of this invention. FIG. 6 is a flow chart of the process summary of the method of designing and manufacturing the artificial joint stem using the composite material of this invention. FIG. 7A shows multiple tomograms, where FIG. 7B is a view showing the condition of reading the form as the two dimensional data and FIG. 7C is a view showing the condition of the element breakdown after making three dimensions. FIG. 8 is a view showing the rough element breakdown of the bone, FIG. 8B is an explanatory view illustrating the method for obtaining the stiffness of the bone, and FIG. 8C is a view showing the detail element breakdown of the internal portion of the bone. Also, FIG. 9A is a graph showing the contact ratio to the cortical bone and the filling ratio in the medullary canal, and FIG. 9B is a graph showing the bending and the tensile stiffness, and FIG. 9C is a graph showing the torsional stiffness.

FIG. 1 shows the artificial joint stem that is designed and manufactured according to the method of designing and manufacturing in this embodiment which is the artificial hip joint stem fixed in the femoris. As shown in FIG. 1, the artificial joint stem in the example is comprised of the composite material, wherein a proximal portion thereof has the neck part 2 to which a spherical head, not shown in the figures, is fixed, and the main part 3 fixed in the femoris and the guide section 4 leading therefrom are positioned around a lower side of the neck 2.

The surface finishing part 5 is formed at the main part 3 of the stem 1, provided with concave-convex on the part of its surface. Further, as shown in the enlarged view of FIG. 4, the chemically bonded layer 6 is formed by impregnating the hydroxyapatite crystal 6a in the plastic film 6b using as the adhesive agent and bonding thereto. By the convexo-concave of the surface finishing part 5, the mechanical bonding is made high between the stem 1 and the insertion hole 8 penetrated into bone 7 for the stem 1 to be embedded. Also, the chemical bonding with bone 7 is made high with the hydroxyapatite crystal 6a which is impregnated in the chemical bonding layer 6 of the surface, allowing the stem 1 to be glued together with the bone 7 more firmly.

As shown in FIG. 2, the internal structure of the stem 1 is configured to have the first external layer 9 with increased torsional stiffness which contacts with the internal surface of the insertion hole 8 penetrated into the bone 7, the main structure layer 10 with its increased bending stiffness which is placed inside the first external layer 9 and is subsequent from the neck part 2 to the main part 3, and the core layer 11 with lower stiffness than the main structure layer 10 and the first external layer 9 that is positioned inside the main structure layer 10, the inner most layer 12 that is placed in between the core layer 11 and the main structure layer 10, and the second external layer 13, which forms the external surface of the guide section 4, with lower stiffness than the structure layer 10 and the first external layer 9.

The composite material used for the stem 1 is the carbon fiber reinforced plastic. As for the carbon fiber, the high modulus, high strength carbon fiber with its elasticity of 200-650 GPa, for example, is used. Also, as for the matrix, the thermoplastic resin, such as polyether ether ketone (PEEK) and polyetherimide (PEI) which are harmless to the human body, is used. Also, the sizing can be applied to the carbon fiber in order to increase the bonding strength to the matrix. Incidentally, as for the stem 1 in the example, if the carbon fiber with its elasticity of 630 GPa used and the layer with its fiber direction ±45° is formed, the layer's transverse modulus G is about 49 GPa, which has enough strength when comparing to the conventional titanium stem of 43.3 GPa.

For the first external layer 9 of the stem 1, the fiber form of the composite material are woven fabric, and the direction of the fibers is directed ±45° to the axis of the main part 3 of the stem 1. As a result, the torsional stiffness increases and the shear loading and the torsional loading that are applied to the stem 9 can be supported at the first external layer 9.

Also, for the main structure layer 10 of the stem 1, the fiber form of the composite material is woven fabric, and the direction of the fibers is directed toward the axis of the main part 3 of the stem 1. As a result, the bending stiffness increases and the bending loading that is applied to the stem 1 can be supported at the main structure layer 10.

As shown in FIG. 2A, this main structure layer 10 is extended from the neck part 2 to the forefront section of the main part 3. That is, it is extended to the boundary between the epiphysis area and the diaphysis area of the bone 7, while the stem 1 is being fixed on the bone 7. Further, the core 11 goes inside of the main structure layer 10 through a given depth from the side of the guide section 4 of the stem 1.

Furthermore, the taper part 14 is formed in the internal edge of the main structure layer 10, as a result of the core layer 11 going into the main structure layer 10. Because of the taper part 14, the thickness in the main structure layer 10 increases, which changes the stiffness of the main structure layer 10, and the main structure layer 10 is structured to decrease its stiffness toward the forefront side.

The core layer 11 of the stem 1 is formed with the low-stiffness material such as plastic foam, and both the inner most layer 12 and the second external layer 13 are made with the low-stiffness material or the layers with its fibers directed at ±45°. The stiffness of the core layer 11 and the second external layer 13 is the minimum required stiffness necessary to insert the stem 1 into the insertion hole 8 in the operation.

As for the stem 1, as shown in the cross section views B1-B6 in FIG. 3, the external form of the stem 1 fits to the internal form of the insertion hole 8 (the medullary canal 8a) penetrated into the bone 7 in most of the cross sections perpendicular to the axis.

Next, the method of designing and manufacturing the stem 1 in this preferred embodiments will be explained in detail with reference to the FIGS. 5-8. For the method of designing and manufacturing the stem 1, this embodiment uses the computer 19, which can be any conventional computers comprising functional structures of an input means 20 having such as a keyboard, a pointing device, and input ports, a central processing unit (CPU) 21, a display such as a cathode ray tube (CRT), and a liquid crystal display (LCD), a printing device such as a printer and a plotter, output means 22 including such as output ports, and a storing device such as RAM, ROM, HDD, FDD, CD/DVD drive for storing programs and data, which are not shown in the figures.

This central processing unit 21 is comprised of a tomographic recognition means 23 for recognizing the tomographic data of input by the input means 20, the three dimensional data means 24 for creating three dimensional data of the bone 7 from the data recognized by the tomographic recognition means 23, the design condition recognition means 25 for recognizing the design condition of the stem 1 of the input by the input means 20, the stress analysis means 26 for analyzing the internal stress and the adhesive stress of the stem 1 and the bone 7 based on the design condition recognized by the design condition recognition means 25 and the three dimensional data created by the three dimensional data means 24, and the analysis result determination means 27 for determining whether the analysis result analyzed by the stress analysis means 26 satisfies the design condition of the analysis result.

Furthermore, the central processing unit 21 is comprised of a stem data creating means 28 that creates the stem data to be the design diagram of the stem 1 for manufacturing when the analysis result determination means 27 judges that the design condition is satisfied, a simulation data creating means 29 for creating the simulation data for performing an operation simulation on the computer 19, a simulation recognition means 30 for recognizing the operation simulation action from the input means 20, and a simulation image creating means 31 for creating simulation image based on information from the simulation recognition means 30 and the simulation data from the simulation data crating means 29.

Furthermore, the central processing unit 21 is comprised of a stem forming data creating means 32 for creating data for controlling the numerical control forming device used to form the model of the stem 1, a data creating means 33 for obtaining material for creating data so as to control the automatic cutter used for obtaining the material of the composite material when forming the stem, a lamination layer support data creating means 35 for creating data to control a lamination layer support display 34 (as shown in FIG. 6) which displays the lamination layer position such as by laser beam when laminating the composite material on the forming die of the stem 1, and an insertion hole processing data creating means 36 for creating data to control the numerical control operation device or the operation support device such as an operation robot (ROBODOC, registered trademark by . . . ) for forming the insertion hole 8 so as to insert the stem 1 in the patient's bone. Furthermore, although omitted in any of the figures, the memory means is also included so as to store the data and the analysis result created by the respective above means in the memory device.

Data from the respective data creating means, such as the three dimensional means 24, the stress analysis means 26, the analysis result determination means 27, and the simulation image creating means 31 of the central processing unit 21 and the stem data, are transmitted to the output means 22 to be displayed such as on the display and the printer and transferred to another devices via the output port.

First of all, the method of designing and manufacturing the stem 1 of the above-described computer 19, as shown in FIG. 6, is to take plural tomographic images 37 of the bone 7 of the patient at which the stem 1 is fixed by using a nondestructive cross section scanner such as CT and MRI (as shown in FIG. 7A), which are input as the tomographic data from the input means 20 of the computer 19 (Step S101). At this time, by using a device, which obtains the tomographic image 20 from difference in transmission speed at the lamination layer, as the nondestructive tomography scanner, the stiffness analysis of the bone 7 can effectively be performed.

Then, when the input tomographic image data is recognized by the tomographic image recognition means 23, the process goes into the step S102 for the three dimension data means 24. In the step S102, as digitalizing the plural input tomographic images 37 to sample the necessary cross section form data 38 of the bone 7 as shown in FIG. 7B, the cross section form data 38 is arranged with imaging intervals, and proximate corrections are conducted on the intervals to create the three dimension data including the internal structure of the bone 7.

Doctors may set appropriate form and stiffness distribution of the stem 1 based on the previous tomographic image 37, three dimensional data image of the bone 7, and the patient's treatment plan (Step S103), which is input in the computer 19 as the design condition (Step S104).

As the design condition is input at the Step S104, the computer 19 recognizes the design condition with the design condition recognition means 25, and analyzes, at the Step S105, such as the internal stress of the stem 1 and the bone 7 adhesive stress of the stem 1 and the bone 7 based on the design condition and the three dimensional data by the stress analysis means 26 using the finite element method.

First, this analysis performs the element breakdown of the bone 7 based on the three dimension data of the bone 7 as shown in FIG. 7C. As shown in FIG. 8A in more detail, rough measure element breakdown is performed and the element breakdown process is performed multiple times to further fine the rough measure element breakdown as shown in FIG. 8C. Then, the stress analysis of the bone is performed as allocating the predetermined value (for example, Young's modulus, etc.) to the respective element. As shown in FIG. 8B, the relation between the bone density and Young's modulus is predetermined, and Young's modulus and the density of the respective element are determined based on the transmission speed obtained from the relation and the nondestructive tomography scanner.

When the analysis result is determined at the Step S105, at the next step S016, whether the analysis result satisfies the design condition input at the step S104 by the analysis result determination means 27. If the design condition is not satisfied, such a result is displayed such as on the display of the output means 22, and the design condition is reconfigured at the Step S103 to input new design condition at the Step S104 to reanalyze at the Step S105.

At the Step S106, if the analysis result is determined to satisfy the design condition, the process goes to the Step S107, and the stem data to be the design drawing of the stem 1 is created based on the analysis result and the design condition by the stem data creating means 28.

When the stem data is created at the Step S107, at the step S108, simulation data for performing the operation simulation on the computer screen of the computer 19 is created based on the data by the simulation data creating means 29, and the image created by the simulation image creating means 31 is displayed based on the data, wherein the doctors operates the input means 20 such as the keyboard and pointing device of the computer 19 as viewing the image to perform the operation of forming the insertion hole 8 of the bone 7 and perform the operation simulation for inserting the stem 1 in the insertion hole 8.

In the next Step S110, if there is a problem in the form of the resulted stem 1 of the simulation, the design condition is reconfigured at the Step S103 to analyze again. On the other hand, if the result of the simulation is preferable, the process goes to the following steps to manufacture the stem 1 based on the stem data.

In the operation, in the case that the insertion hole 8 of the stem 1 is formed by operation support devices such as the operation robot and numerical control operation device, in the Step S111, the insertion hole forming data is created as the control data by the insertion hole forming data creating means 36.

In the Step S112, the stem forming data is created by the stem forming data creating means 32 using the stem data so as to form the model of the stem 1 by a light emitting device, and the data is transmitted to the light emitting device via the output means 22 to form the model of the stem 1. Next, at the Step S114, the stem 1 is made by the reverse moulage using plaster or resin moulage based on the formed model of the stem 1. Also, the forming die is preferably a segmental die such as being divided into two or three.

At the Step S115, the computer 19 creates the data for obtaining the material so as to obtain the material as controlling the automatic cutter, which is not shown in the figures, and as cutting the raw material of the composite material based on the stem data by the data creating means 33 and obtains the material as transmitting the data to the automatic cutter via the output means 22 and as cutting the raw material of the composite material (Step S116). Also, for the raw material of the composite material, the reinforced fiber such as the carbon fiber and the fiber made of thermoplastic resin proving matrix are preferably used to make textiles.

In the Step S117, the lamination layer support data is created to display the lamination layer position of the composite material on the forming die of the stem 1 by the lamination layer data creating means 35 using the lamination support display 34, and the data is transmitted to the lamination layer support display 34 via the output means 22.

Furthermore, in the Step S118, the material such as the composite material is layered on the forming die of the stem 1. In detail, the position of the surface treatment portion 5 is displayed on the forming die by the lamination layer support display 34, and the resin sheet with impregnated hydroxyapatite crystal is arranged on the subject position. In the next step, the raw material of the composite material after obtaining the material at the Step S116 is layered according to the displayed instruction on the lamination layer support display 34. Here, the layered raw material becomes the first external layer 9 after the formation, and the direction of the reinforced fiber is ±45° relative to the axial direction of the stem 1, wherein the direction of the reinforced fiber is such that the automatic cutter precuts the reinforced fiber while fixing the direction thereof to make a desirable direction when layered on the forming die.

Next, the raw material of the composite material for forming the main structure layer 10 is layered. This raw material has the same form as above-example and is precut by the automatic cutter so as to direct the reinforcement fiber in the axial direction of the stem 1. The raw material that forms the inner most layer 12 and the second external layer 13 is arranged, and the foamed material to make the core layer 11 is arranged in a space formed by the inner most layer 12 and the second external layer 13.

In the Step S118, when the layering of such as the composite material is completed, in the Step S119, segmented forming die is closed to heat and press thereof for a certain period of time by the hot plate and autoclave. At this time, the thermoplastic resin is dissolved to impregnate into the textile made of the reinforcement fiber to make matrix. Also, the above-laying process may be performed while increasing the flexibility of the thermoplastic resin such as in the heated space. Thereafter, the stem 1 is cooled down to the predetermined temperature and is removed from the forming die. Furthermore, the Step S120 is to finish the burr of the formed stem 1, and the next Step S121 is to final check the stem 1 to complete the stem 1.

Thereafter, in the operation, the insertion hole 8 is formed in the bone 7 such as by the operation robot and the stem 1 is inserted to be fixed therein based on the insertion hole forming data created in the Step S111. In the Step S109, the doctor who performs operation has done the simulation of inserting the stem 1, which the process of inserting and fixing the stem 1 (Step S122).

As shown in FIG. 9A, while the stem 1 manufactured according to the above-described example of the method of designing and manufacturing has the low contact ratio to the cortical bone and the filling ratio in the medullary canal, that is fit and fill, near the opening of the insertion hole 8, the fit and fill is higher in the more forefront side, and undergoes the transition at about 70% contact ratio to the cortical bone and filling ratio in the medullary canal all the way to the forefront side (side of the guide section 4).

FIG. 9A is the contact ratio to the cortical bone and filling ratio in the medullary canal shown in the form of a graph (solid line), and its contact ratio to the cortical bone and the filling ratio in the medullary canal are significantly higher than the conventional cement-less type stem (a dashed lines) and the custom made stem (dashed lines) in which the conventional cement-less type stem is improved. That is, the fit and fill of the stem 1 is generally high in the main part 3 and the guide section 4. The reference number 15 in the figure is the area where the main body 3, in which the taper part 14 is not provided, is located. The reference number 16 is the area where the taper part 14 of the main part 3 is provided. The reference number 17 is the area where the guide section 4 is located.

However, as shown in FIG. 9B and FIG. 9C of the same figure, in the epiphysis and the diaphysis area, that is the part in the main structure layer 10 of the stem 1 where the taper part 14 is provided, the bending and tensile stiffness are quickly decreasing and the torsional stiffness is gradually decreasing, as getting toward the forefront side (the side of the guide section 4) of the stem 1. As a result, because the stiffness of the guide section 4 is low although the overall fit and fill is high, and the stem's loading is transferred to the bone 7 through the high-stiffness main part 3, the proximal fixing of the stem 1 is possible.

This is also illustrated in FIG. 3. To elaborate, from this cross section, in the main part 3, the main structure layer 10 is mainly occupied, and the bending and tensile stiffness is granted by the main structure layer 10 and the first external layer 9 outside of it. And the low-stiffness core layer 11 and the internal layer 12 are expanded to the center of the stem 1 as getting from the main part 3 to the guide section 4, and there are only low-stiffness core layer 11 and the second external layer 13 at the guide section 4. From this, we know that the loading of the stem 1 is largely transferred to bone 7 at the main part 3.

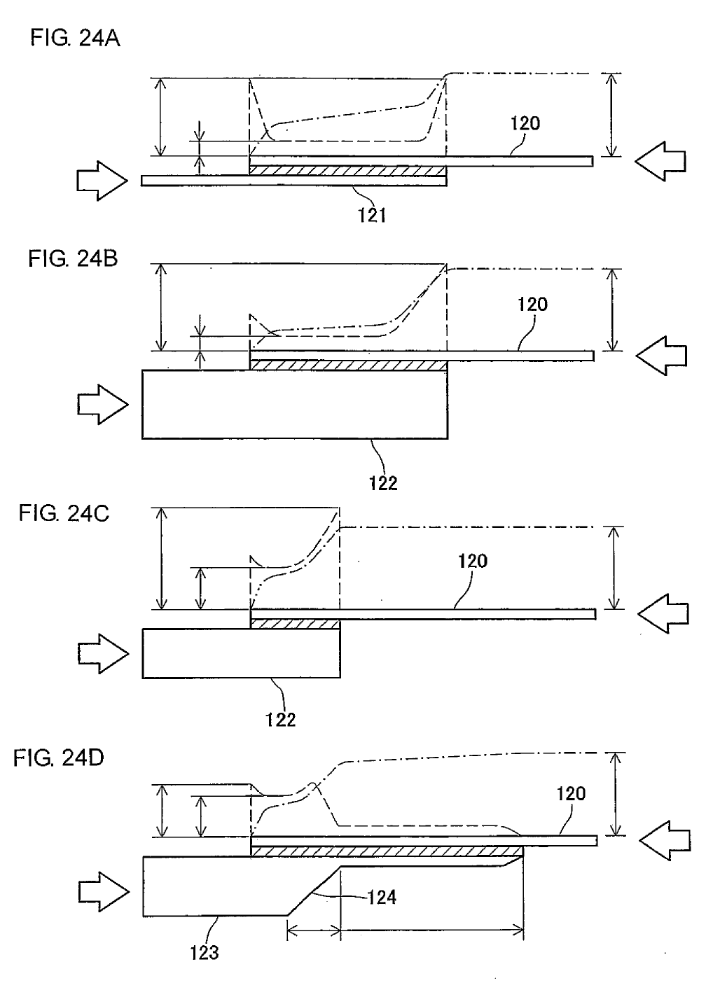

The load transfer concept between the stem 1 and the bone 7 is the same as the one shown in FIG. 24(D), thereby being designed and manufactured to restrain the stress concentration on the both ends of the contact layer of the bone 7.

As such, this embodiment makes it possible to design and manufacture the stem 1 with the form and stiffness corresponding to the form and structure of the patient's bone 7 by using the computer 19. Accordingly, improving the fit and fill between the stem 1 and the bone 7 enables the initial fixation, and because the rotational fixation is high, an early discharge from hospital is possible through shortening the hospitalization period, and an early social rehabilitation is possible and thus relieving the burden on the patient. Also, this method can be utilized for senior people, who have concerns about adverse effect of motor functions and other functions resulting from a long-term hospitalization.

Also, because of the improved fit and fill, the stem 1 can be well connected to bone 7 without cement, and there is no adverse effect on the human body through the melt-out of the unreacted monomer from not being mixed enough or the mixture ratio is inaccurate.

Also, because the fit and fill can be improved and the load from the stem 1 can be transferred to the bone without deviation, the stress shielding can be controlled, thereby making the bone 7 thinner and weakening the connection between the stem 1 and the bone 7, which prevents loosening the stem 1 and allows to design and manufacture the stem 1 with high durability.

Also, the computer 19 is used to perform the complicated three dimension stress analysis using the finite element method, which enables to shorten the time necessary for this analysis dramatically, shorten the time necessary for the manufacturing process dramatically, and also reduces the burden on the patient with respect to the hospitalization.

Furthermore, the composite material is used as the stem 1, in particular, by using the composite material that is harmless to the human body, there is no adverse affect to the human body unlike the conventional metallic stem in which the harmful substance to the human body melts out from the stem to the inside of human body. Also, the composite material is excellent in formability and workability compared to the titanium alloy, and the stem 1 is formed using the forming die based on the stem data, which facilitates to obtain the desirable form and high accuracy, thereby reducing the cost and shortening the time for manufacturing the stem 1.

Also, because the forming data of the insertion hole 8 formed at the bone 7 is created by the stem data identical to the data for forming the forming die of the stem 1, the internal surface form and the external surface form of the insertion hole 8 can be matched as much as possible.

Furthermore, the forefront side of the stem 1 has the guide section 4, and the simulation of inserting the stem 1 can be performed on the computer 19, which allows to sufficiently perform the simulation, thereby facilitating the insertion process of the stem 1 in the insertion hole 8 formed in the bone 7 at the actual operation.

Furthermore, when forming the stem 1, such as the automatic cutter 34 and the lamination layer support display 34 are used to obtain the material of the composite material and to determine the lamination position of the composite material, which effectively avoid mistakes of the operator and increases the reliability of manufactured stem 1.

So far, we have illustrated the various embodiments of the invention, yet the invention is not limited to these embodiments, and various improvements as well as changes of design are possible to the extent it does not deviate from the scope of the invention, as indicated below.

That is, as the stem 1 manufactured according to the above-method of designing and manufacturing the artificial joint stem, the clearance between the external form of the stem 1 including the guide section 4 and the internal form of the insertion hole 8 is designed to be minimized; however, this invention is not limited thereto, wherein in order to increase the proximal fixing of the stem, the guide section 4 may be designed such that the predetermined amount of clearance may be designed to be formed between the external surface and the inner surface of the insertion hole 8.

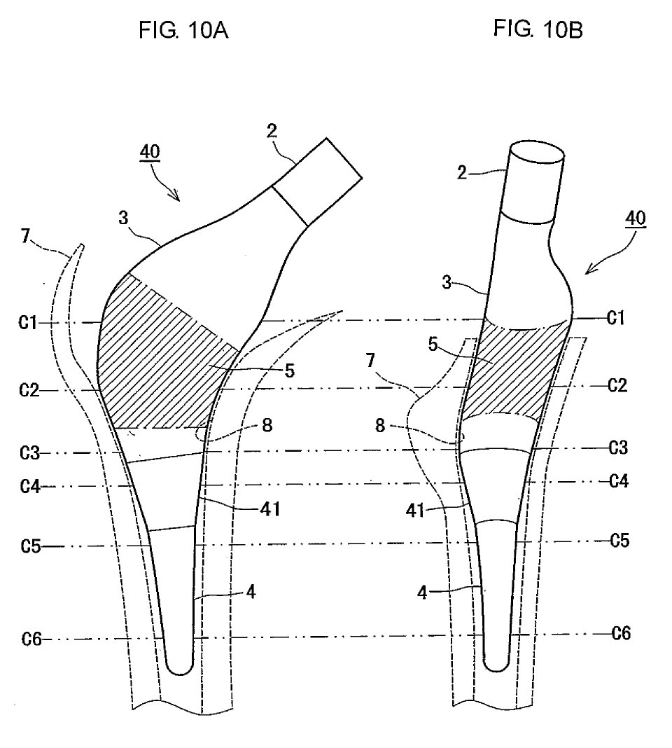

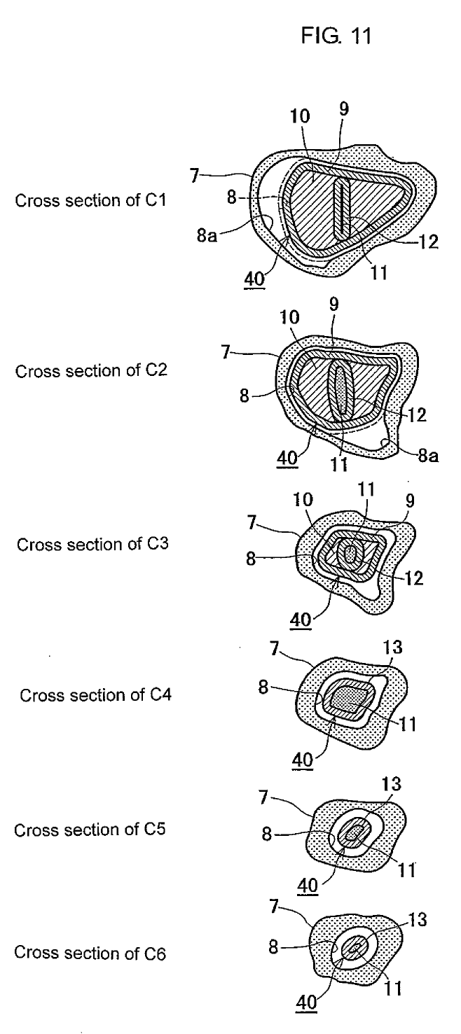

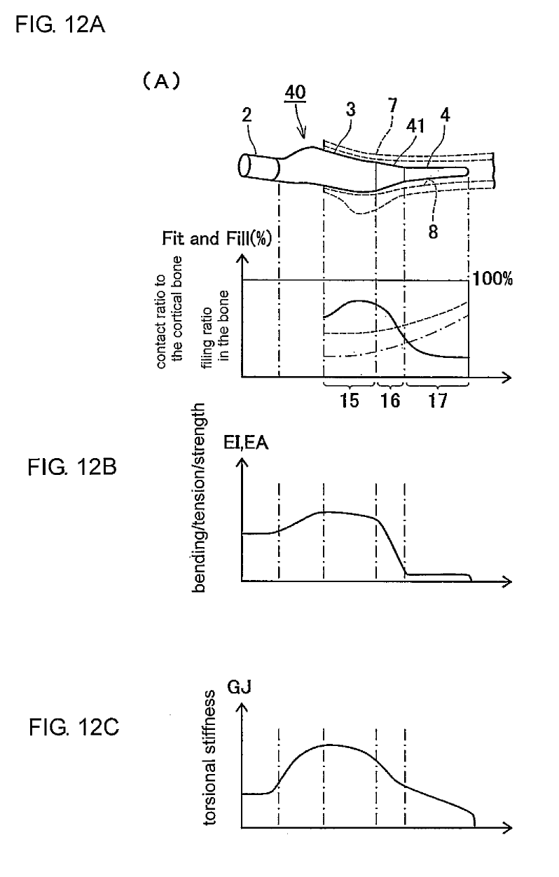

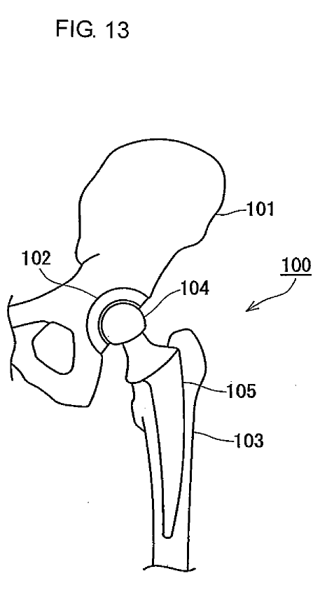

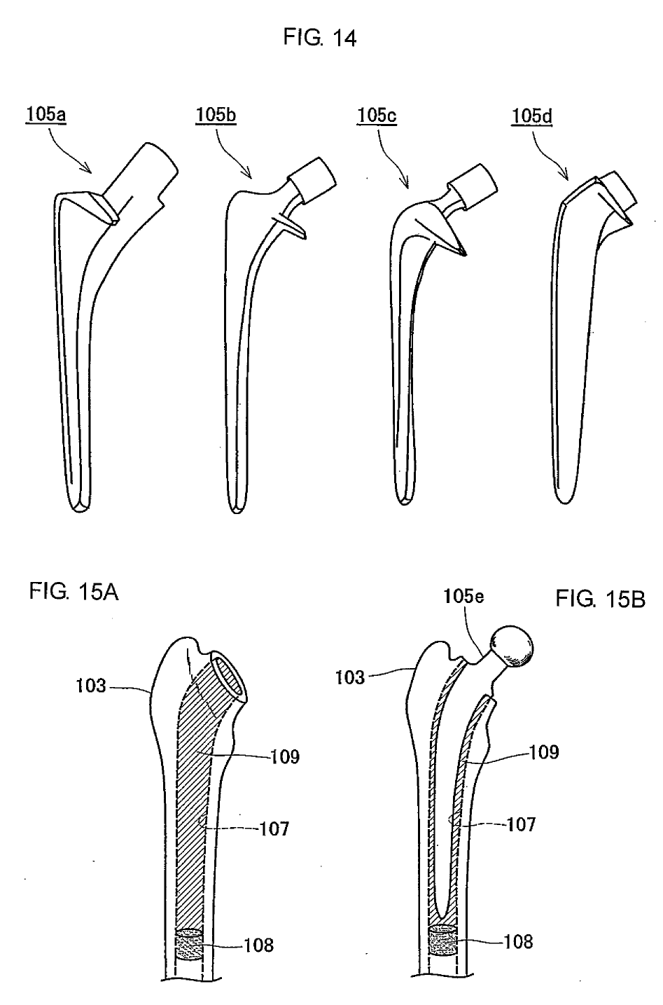

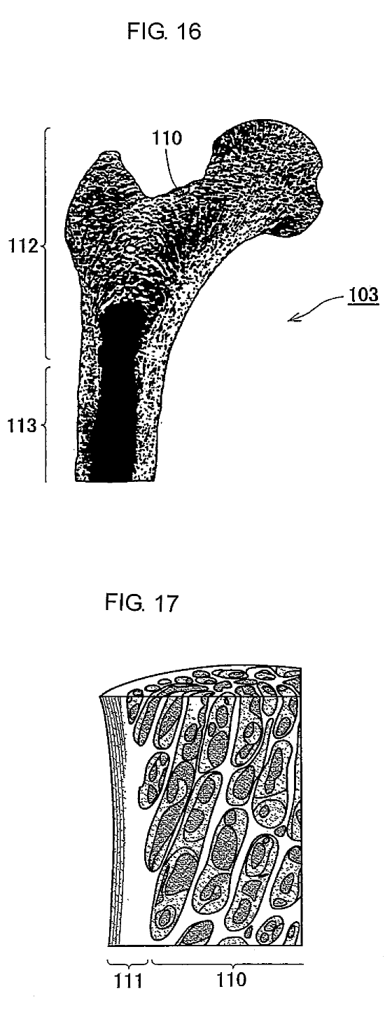

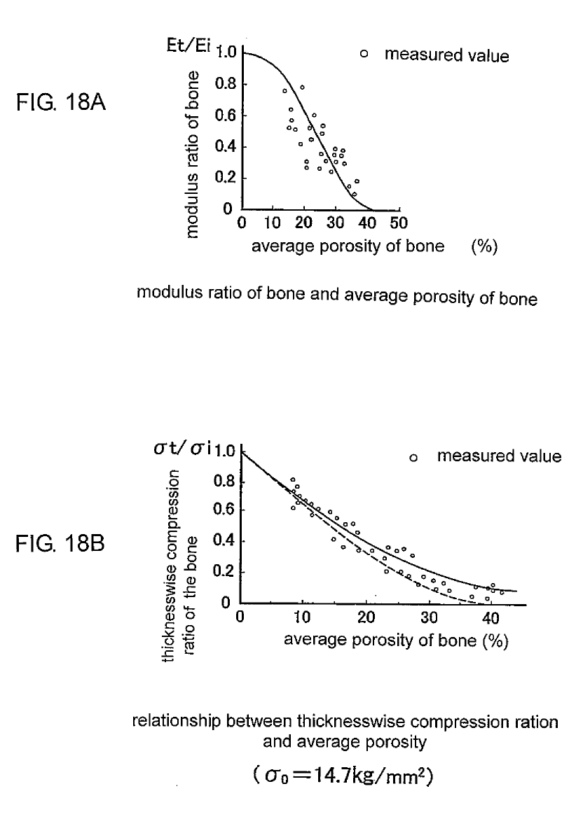

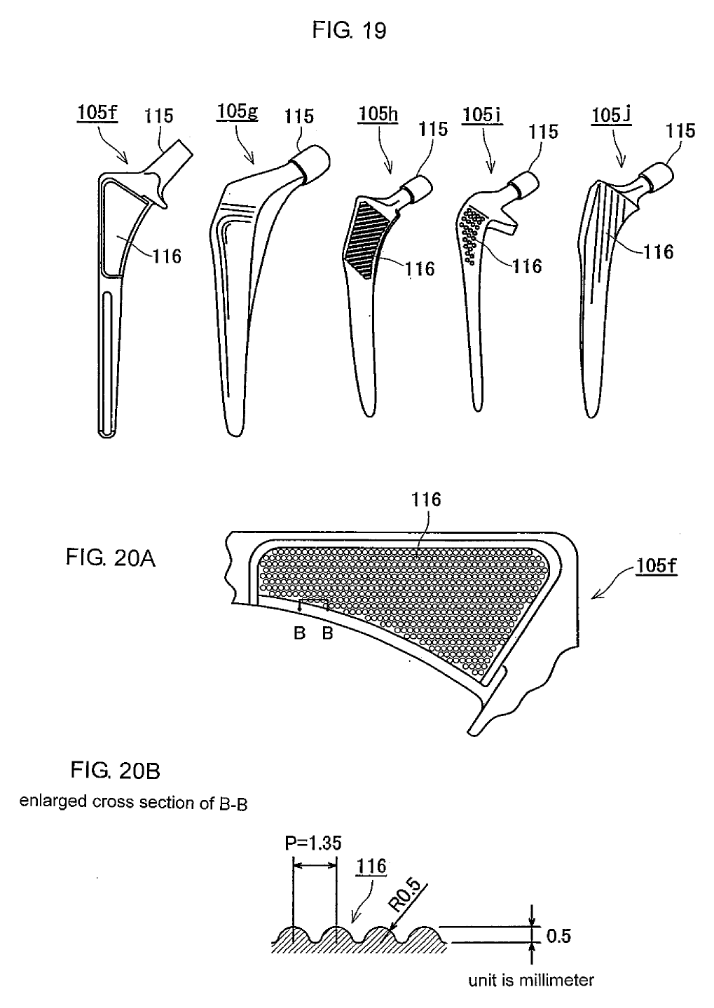

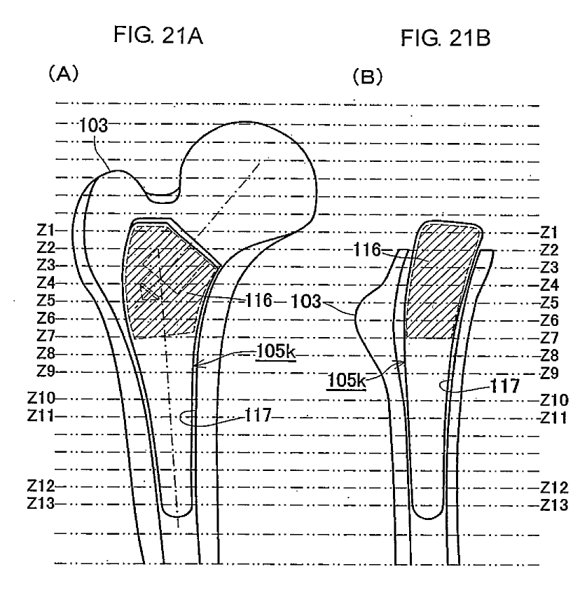

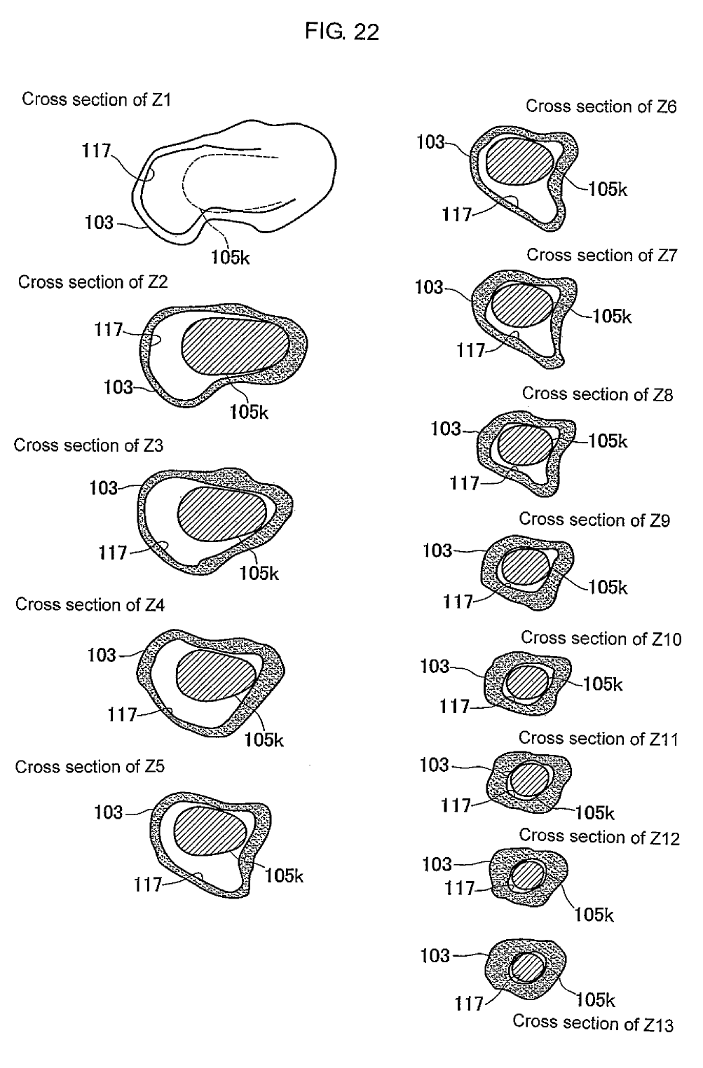

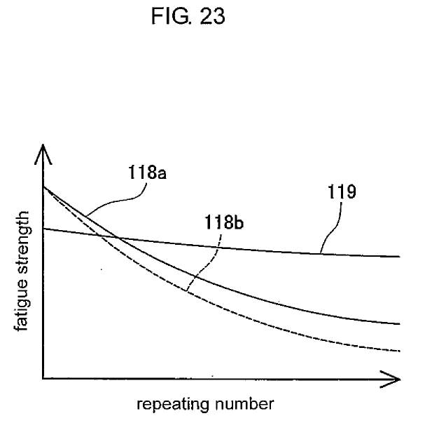

One embodiment of the artificial joint stem of such a kind will be explained with reference to FIGS. 10-12. FIG. 10A is a front view of the artificial joint stem different from the one in FIG. 1 manufactured with the use of the method of designing and manufacturing the artificial joint stem using the composite material in the invention, and FIG. 1B is its side view thereof: FIG. 11 is a set of cross section views of C1-C6 in FIG. 10 that are cut in each level perpendicular to the axes; Also, FIG. 12A is a graph showing the contact ratio to the cortical bone and the filling ratio in the medullary canal, and FIG. 12B is a graph showing the bending and the tensile stiffness, and FIG. 12C is a graph showing the torsional stiffness. As for the parts similar to the abovementioned example, the same reference signs are provided and the illustration of which is omitted.

The stem 40 in this embodiment has a high fit and fill at the main part 3, that is, in the epiphysis area, and a low fit and fill at the guide section 4, that is, in the diaphysis area, making a perfect anchorage between the stem 40 and the bone 7 in the epiphysis area, that is, the proximal fixing.

As shown in FIG. 10 and FIG. 11, the taper part 41 is provided between the main part 3 and the guide section 4 for the stem 40 in this example, and the given amount of clearance is formed between the outer surface of the guide section 4 and the internal surface of the insertion hole 8, as a result of the external form of the guide section 4 being smaller by the taper part 41.

From this, as shown in FIG. 12A, while the contact ratio to the cortical bone and the filling ratio in the medullary canal (fit and fill) are high in the main part 3 of the stem 40, the fit and fill decreases in the taper part 41, and the fit and fill for the guide section 4 remains low through the forefront.

As such, according to the method of designing and manufacturing in this embodiment, since the appropriate amount of clearance is formed between the external surface of the guide section of the stem 40 and the internal surface of the insertion hole 8, the guide section 4 does not contact with bone 7 in the early postoperative period, thereby the loading is not transferred to bone 7 through the guide section 4.

Also, after the surgery, even if the clearance with the guide section 4 is filled due to the growth of the bone 7, this part is filled with the low density cancellous bone, and the stress applied to the joint section with the guide section 4 is small, and the loading from the stem 40 is largely applied in the epiphysis area where the main part 3 is located. The anchorage in the epiphysis area is continuously maintained, and thus the loading from the stem 40 can be transferred to the bone 7 in a good condition.

Furthermore, as for the stem 40 in this example, since the guide section 4 is thin, the friction of the guiding 4 is low when the stem 40 is inserted into the insertion hole 8 during the surgery, and thus the insertion can be done more easily than the stem 1 in FIG. 1.

Furthermore, although the stems 1, 40 manufactured according to the method of designing and manufacturing the above-artificial joint stem has the guide section 4, the stem is not limited thereto, and the guide section 4 is not a requirement. According to the method of designing and manufacturing, simulation for inserting the stems 1, 40 on the computer 19 can be done prior to the actual operation, and therefore it is possible to learn the inserting feeling by the simulation, thereby facilitating the insertion of the stems 1, 40 in the insertion hole 8 without the guide section 4.

Also, in the above method of designing and manufacturing the artificial joint stem, the insertion hole forming data is created to control the operation robot based on the stem data; however, this invention is not limited thereto, and the broach cutter is provided as a cutting tool for forming the insertion hole 8 so as to form the insertion hole using the broach cutter based on the stem data.

Furthermore, in the above method of designing and manufacturing the artificial joint stem, after making the stem model by the light forming device based on the stem data, the forming die is made from the model using the plaster or resin moulage; however, this invention is not limited thereto, and for example, such as NC data is created based on the stem data by the numerical control forming device to form a direct forming die. Accordingly, this invention can reduce the cost and time for manufacturing without the model. Furthermore, for the raw material of forming die, metal such as aluminum alloy and fusible alloy, inorganic material such as plaster and potassium silicate, and organic material such as resin can be used as examples, and the raw material is preferably durable at temperature in the formation period and provides easy finishing after cutting.

Also, the above described method of designing and manufacturing the artificial joint stem disclosed that the computer 19 is comprised of data creating means such as the stem forming data creating means 32, data creating means to obtain the material 33, the lamination layer support data creating means 35, and the insertion forming data creating means 36; however, this invention is not limited thereto and these means can be provided in other computers or numerical control forming device.

### INDUSTRIAL APPLICABILITY

In addition to the artificial joint stem of femur as the above described embodiments, this invention may be used in designing and manufacturing implant to connect joints such as knee joint, shoulder joint, and fractured bone or for the substitute of damaged bone by accident or disease.

1. A method of designing and manufacturing an artificial joint stem with the use of a composite material comprising:
a first external layer, which is inserted and fixed in an insertion hole formed in a bone without filling cement, torsional stiffness thereof is increased as contacting an internal surface of said insertion hole;
a main structure layer, which is positioned in an inner side than said first external layer, bending stiffness thereof is increased;
a core layer with lower stiffness than the main structure layer and the first external layer, which is positioned in an inner side than said main structure layer; and
a most inner layer, which is positioned between the core layer and the main structure layer;
steps of performing, as using a computer, an analysis involving an internal stress of said artificial joint stem and an adhesive stress of said artificial joint stem and the bone based on three dimension data indicating a structure of said bone made by using plural bone tomographic images and a design condition involving a form and stiffness of said artificial joint stem configured at least by one of said tomographic images and said three dimension image;
having said computer to reanalyze as changing said design condition if a result of said analysis fails to satisfy said design condition; designing and manufacturing said artificial joint stem using stem data based on said result of said analysis and said design condition if said result of said analysis satisfies said design condition.

2. The method of designing and manufacturing the artificial joint stem with use composite materials according to claim 1, further comprising step of performing analysis including the internal stress of the bone analysis using the finite element.

3. The method of designing and manufacturing the artificial joint stem which uses composite materials according to claim 2, wherein said tomographic image is a tomographic image which is obtained by different transmission speed of the layers of the bone, and further comprising a step of analyzing the internal stress of the bone as determining the Young's modulus and the density of every element of the bone based on the relation of the predetermined density and Young's modulus of the bone and the transmission speed.

4. The method of designing and manufacturing artificial joint stem which uses composite materials according to claim 1, further comprising a step of forming as superposing the composite materials of the first external layer, the main structure layer, and the core layer by molding in a die.

5. The method of designing and manufacturing artificial joint stem which uses the composite materials according to claim 1, further comprising a step of forming a model of said artificial joint stem or a forming die.

6. The method of designing and manufacturing artificial joint stem which uses the composite materials according to claim 1, further comprising a step of obtaining a material of the composite materials for the use in forming said artificial joint stem as controlling an automatic cutter based on the stem data.

7. The method of designing and manufacturing artificial joint stem which uses the composite materials according to claim 1, further comprising a step of displaying a lamination layer position of the composite materials used in forming said artificial joint stem in a forming die of said artificial joint stem based on the stem data.
